SEQUENCE LISTING (16 sequences)

SEQ ID NO: 1 - protein, 100 aa, Oryza sativa
MNSTSHFVAISFFHHQLDQTAETREDELVGEDVVAGGGWAEHRGGQEGDSQGQARRRHRRAAIRHGGARGFSLQPCPNLRRHRRRDASCGLASRIYIALN

SEQ ID NO: 2 - DNA, 303 bp, Oryza sativa
  source 1..303: mol_type = genomic DNA
atgaactctacaagtcattttgtcgccatcagcttctttcatcaccaactcgatcagaccgcagagacgagggaagatgagctcgtcggcgaagacgtcgtggccggaggtggttgggctgagcatagaggaggccaagaaggtgattctcaaggacaagcccgacgccgacatcgtcgtgctgccattcggcacggcggtgccagaggattttcgcttcaaccgtgtccgaatcttcgtcgacaccgtcgccgagacgcctcgtgtgggctagctagcagaatatatatagctttaaattaa

SEQ ID NO: 3 - DNA, 23 bp, synthetic construct
  source 1..23: mol_type = other DNA
ggcacgagggaagatgagctcgt

SEQ ID NO: 4 - DNA, 23 bp, synthetic construct
  source 1..23: mol_type = other DNA
aaacacgagctcatcttccctcg

SEQ ID NO: 5 - DNA, 22 bp, synthetic construct
  source 1..22: mol_type = other DNA
ctccgttttacctgtggaatcg

SEQ ID NO: 6 - DNA, 20 bp, synthetic construct
  source 1..20: mol_type = other DNA
cggaggaaaattccatccac

SEQ ID NO: 7 - DNA, 38 bp, synthetic construct
  source 1..38: mol_type = other DNA
ttcagaggtctctctcgcactggaatcggcagcaaagg

SEQ ID NO: 8 - DNA, 37 bp, synthetic construct
  source 1..37: mol_type = other DNA
agcgtgggtctcgaccgggtccatccactccaagctc

SEQ ID NO: 9 - DNA, 47 bp, synthetic construct
  source 1..47: mol_type = other DNA
gttacttctgcactaggtaccatgaactctacaagtcattttgtcgc

SEQ ID NO: 10 - DNA, 55 bp, synthetic construct
  source 1..55: mol_type = other DNA
tcttagaattcccggggatccttaatttaaagctatatatattctgctagctagc

SEQ ID NO: 11 - DNA, 20 bp, synthetic construct
  source 1..20: mol_type = other DNA
aagaagctgaagcatccagc

SEQ ID NO: 12 - DNA, 20 bp, synthetic construct
  source 1..20: mol_type = other DNA
ccaggacaagatgatctgcc

SEQ ID NO: 13 - DNA, 23 bp, synthetic construct
  source 1..23: mol_type = other DNA
acgacccgtctttacttatttgg

SEQ ID NO: 14 - DNA, 23 bp, synthetic construct
  source 1..23: mol_type = other DNA
aagtagcgttggttttgttggat

SEQ ID NO: 15 - DNA, 46 bp, synthetic construct
  source 1..46: mol_type = other DNA
gatctggttccgcgtggatccaactctacaagtcattttgtcgcca

SEQ ID NO: 16 - DNA, 55 bp, synthetic construct
  source 1..55: mol_type = other DNA
ctcgagtcgacccgggaattcttaatttaaagctatatatattctgctagctagc

(19) United States
(12) Patent Application Publication
(10) Pub. No.: US 2024/0002875 A1
(43) Pub. Date: Jan. 4, 2024

(54) Use of rice OsPR6 gene or protein encoded thereby in regulating resistance of rice to Magnaporthe oryzae

(71) Applicant: CHINA NATIONAL RICE RESEARCH INSTITUTE, Hangzhou (CN)

(72) Inventors: Yanjun KOU, Hangzhou (CN); Ya CHEN, Hangzhou (CN); Jiehua QIU, Hangzhou (CN); Huanbin SHI, Hangzhou (CN)

(21) Appl. No.: 18/340,910

(22) Filed: Jun. 26, 2023

(30) Foreign Application Priority Data
Jun. 29, 2022 (CN) 202210763723.1

(51) Int. Cl. C12N 15/82

(52) U.S. Cl. CPC C12N 15/8282

(57) ABSTRACT

The present disclosure discloses use of a rice OsPR6 gene or a protein encoded thereby in regulating resistance of rice to Magnaporthe oryzae, and relates to the technical field of prevention and treatment of rice blast. A CDS nucleotide sequence of the rice OsPR6 gene is shown in SEQ ID NO. 2. A study finds that the rice gene OsPR6 plays an important role in a resistance process of rice to rice blast. The gene may be used for screening to obtain a rice plant line resistant to Magnaporthe oryzae, or the protein encoded thereby may be used as a drug for preventing and treating rice blast.

### CROSS-REFERENCES TO RELATED APPLICATION

The present application claims the benefit of the priority of the Chinese patent application with the application CN202210763723.1, filed to the China National Intellectual Property Administration on Jun. 29, 2022, the entire content of which is incorporated in this application by reference.

### SEQUENCE LISTING

The present application contains a Sequence Listing which has been submitted electronically in ASCII format and is hereby incorporated by reference in its entirety. Said ASCII copy is named_Sequence_Listing.xml and is 15,516 bytes in size, and the Sequence listing is identical to the priority of the Chinese patent application CN202210763723.1, filed to the China National Intellectual Property Administration on Jun. 29, 2022.

### TECHNICAL FIELD

The present disclosure relates to the technical field of prevention and control technology of rice blast, and particularly relates to use of a rice OsPR6 gene or a protein encoded thereby in regulating resistance of rice to Magnaporthe oryzae.

### BACKGROUND

A plant faces a variety of pathogen infection threats in a natural ecosystem, leading to reduced yield and quality in agricultural production. A plant disease mainly includes a fungal disease, a bacterial disease, a viral disease, etc. Diseases of an important monocotyledonous food crop, rice, include a most representative fungal disease such as rice blast caused by infection with Magnaporthe oryzae; a bacterial disease such as bacterial bight caused by infection with Xanthomonas oryzae pv oryzae; and a viral disease such as a rice stripe virus disease caused by infection with a rice stripe virus. A common fungal disease infecting a dicotyledonous crop or a vegetable includes a rotten disease, etc. caused by infection with B. cinerea. The rice blast may occur in different growth and development stages and at different parts of rice, mainly including leaf blast, neck blast, and grain blast. The Magnaporthe oryzae is propagated in the field mainly in an asexual state, and completes an infection cycle. Three-celled conidiospores of Magnaporthe oryzae scattered on weeds or diseased and disabled plants in a field in the previous year are scattered on surfaces of rice leaves along with air, rainwater, etc. Cells at top ends of the conidiospores release mucus and are tightly attached to the surfaces of the hydrophobic rice leaves, and the conidiospores are prevented from falling off under the action of an external force such as rainwater. Then self nutrient substances are consumed by the conidiospores within 1-2 h, and the conidiospores germinate to form a narrow germ tube extending along the surfaces of the rice leaves. The germ tube stops growing after extending for a certain distance on the surfaces of the rice, and expands and differentiates at the top ends to form a specialized appressorium with an infection structure. A single-celled appressorium gradually matures, and the three-celled conidiospores undergo an autophagy death and transport energy and substances to the appressorium. Then the appressorium gradually expands to generate a turgor pressure of about 8 MPa under an accumulation action of glycerol and melanin, and a special structure of “an infection plug” is formed at a base part to pierce through a cuticle of rice leaves and invades host cells. A primary infected hypha may be differentiated in the rice cells to generate a secondary infected hypha which is expanded among the rice cells. Under a humid environment, grey or grayish brown spindle disease spots appear on the rice leaves after the Magnaporthe oryzae infection for 72-96 h. In a process, the Magnaporthe oryzae is changed from a living body nutrition type to a dead body nutrition type. Newly generated conidiospores at the disease spots may be spread to a new host plant through wind and rain, and a new round of infection is started.

An influence of the diseases on yield and quality of crops or vegetables is the most serious. A yield reduction caused by the diseases each year is equivalent to 10-30% of the total yield. The use of a pesticide reduces the influence of the diseases, but there are adverse factors such as pesticide residues and negative influence on the natural environment. Therefore, breeding for disease resistance is a most economical and effective method for resisting pathogens at present. Exploring a new rice blast disease-resistant gene and resistant resource, and cultivating a broad-spectrum resistance variety are a green and efficient strategy for guaranteeing yield and quality of crops, and have important significance for agricultural production.

However, few novel drugs effective in preventing and controlling rice blast are currently known.

### SUMMARY

The disclosure discloses a regulation and control mechanism of a rice disease resistance from a molecular level, finds a new disease-resistant related gene, and uses the gene to inhibit formation of appressorium of Magnaporthe oryzae. The gene is tried to be used in preventing and treating rice blast, and developing a drug for efficiently preventing and treating the rice blast.

A study of the present disclosure finds that a rice OsPR6 gene is related to an immunoreaction of rice to rice blast. After the OsPR6 gene is knocked out, a defense capability of the rice to the Magneporthe oryzae is reduced. But over-expressing the OsPR6 gene may improve a disease resistance of the rice to the Magnaporthe oryzae. Besides, an OsPR6 protein has an obvious inhibition effect on formation of appressorium of Magnaporthe oryzae and may be used as a drug for preventing and treating rice blast.

The technical solution of the present disclosure is as follows:

The present disclosure provides use of a rice OsPR6 gene or a protein encoded thereby in regulating resistance of rice to Magneporthe oryzae.

The present disclosure further provides use of a rice OsPR6 gene or a protein encoded thereby in preparing a drug for resisting Magnaporthe oryzae.

An OsPR6 purified protein (PR6GST protein) has an obvious inhibiting effect on appressorium of Magnaporthe oryzae. When the concentration of the PR6GST is 0.1 μg/μL, a formation rate of the appressorium of Magnaporthe oryzae is only 23.97%, but when the concentration of the PR6GST reaches 0.2 μg/μL, germination of Magnaporthe oryzae spores is extremely obviously inhibited, and no appressorium is formed.

Preferably, a CDS nucleotide sequence of the rice OsPR6 gene is shown in SEQ ID NO. 2, and an amino acid sequence of the protein encoded by the rice OsPR6 gene is shown in SEQ ID NO. 1.

The present disclosure further provides a method for regulating resistance of rice to Magnaporthe oryzae. When the resistance of rice to Magnaporthe oryzae needs to be reduced, an OsPR6 gene in rice is silenced or knocked out; and when the resistance of rice to Magnaporthe oryzae needs to be improved, the OsPR6 gene in rice is overexpressed.

The present disclosure further provides use of a rice OsPR6 gene in rice breeding, wherein a rice plant line resisting Magnaporthe oryzae is obtained by screening a rice plant with a high expression of the rice OsPR6 gene.

The present disclosure further provides use of a protein encoded by a rice OsPR6 gene in rice breeding, wherein a rice plant line resisting Magnaporthe oryzae is obtained by screening a rice plant with a high expression level of the protein encoded by the rice OsPR6 gene.

The present disclosure further provides a drug for resisting Magnaporthe oryzae, wherein an active component includes a rice OsPR6 gene with a CDS nucleotide sequence shown in SEQ ID NO. 2 or a protein encoded by the rice OsPR6 gene with an amino acid sequence shown in SEQ ID NO. 1.

The present disclosure further provides use of the drug for resisting Magnaporthe oryzae in controlling and treating Magnaporthe oryzae infection.

The present disclosure further provides a method for constructing a transgenic rice resisting Magnaporthe oryzae, wherein a rice OsPR6 gene is transferred into a rice plant to obtain a transgenic rice with a high expression of the rice OsPR6 gene.

Specifically, a nucleotide sequence of a CDS region of the rice OsPR6 gene is cloned into a vector, and the vector is firstly transferred into Agrobacterium, and then transferred into a rice cell by a callus transformation to obtain a transgenic rice with a high expression of the rice OsPR6 gene.

A study of the present disclosure finds that the rice gene OsPR6 plays an important role in a resistance process of rice to rice blast. The gene may be used for screening to obtain a rice plant line resistant to Magnaporthe oryzae, or the protein encoded thereby may be used as a drug for preventing and treating rice blast.

### BRIEF DESCRIPTION OF THE DRAWINGS

FIG. 1 shows an identification result of rice blast resistance of rice OsPR6 knockout and overexpression mutants, wherein A shows in-vitro inoculation; and B shows relative hyphal biomass of diseased leaf, * p<0.05, ** p<0.01, and *** p<0.001.

FIG. 2 shows a detection result of purification of rice OsPR6 protein.

FIG. 3 shows a result of inhibiting formation of appressorium of Magnaporthe oryzae by rice OsPR6 purified protein, wherein A shows formation of appressorium of Magnaporthe oryzae, and B shows a statistical result of formation of appressorium of Magnaporthe oryzae.

### DETAILED DESCRIPTION OF THE EMBODIMENTS

### Example 1

Rice OsPR6 gene knockout mutants were obtained.

An amino acid sequence of a rice OsPR6 protein was shown in SEQ ID NO. 1 and a gene sequence (CDS sequence) of a rice OsPR6 gene was shown in SEQ ID NO. 2. A target gene was directionally knockout by a Crispr/Cas9 technology, so as to obtain an OsPR6 gene knockout mutant.

A knockout vector was constructed by the following steps:
(1) Firstly, a reference sequence “http://rice.uga.edu/cgi-bin/sequence_display.cgi?orf=LOC_Os12g25090.1” in Nipponbare was downloaded according to the gene number of OsPR6, a target site was determined, and linker primers OsPR6cas9-F/R were designed. Primer sequences were as follows:

OsPR6cas9-F (SEQ ID NO. 3):
ggcACGAGGGAAGATGAGCTCGT;
and
OsPR6cas9-R (SEQ ID NO. 4):
aaacACGAGCTCATCTTCCCTCG.

(2) Preparation of target linker: the linker primers were dissolved into a 100 μM mother solution, 1 μL of each mother solution was added into 98 μL of ddH₂O to be mixed, and the mixture was diluted to 1 μM, treated at 90° C. for 30 s, and then moved to room temperature to cool for 20 min to finish annealing.
(3) Ligation reaction of gRNA expression cassette: an enzymatically digested pYL gRNA-U3 vector was ligated to a corresponding target linker. A PCR reaction system was as follows:

| pYLgRNA-U3 | 0.5 μL (~10 ng), | |
|---|---|---|
| Target linker (final concentration 0.1 μM) | 1 | μL, |
| Bsal (20 U/μL) | 0.25 | μL, |
| 10 × T4 DNA ligase buffer | 1 | μL, |
| T4 DNA ligase | 0.1 | μL, |

| | |
|---|---|
| ddH₂O | supplemented to 10 μL; and |

PCR reaction conditions:
37° C., 5 min; and 20° C., 5 min. A total of 5 cycles.
(4) Amplification of gRNA expression cassette (two rounds of nested PCR amplification):

The ligated product in (3) was subjected to a first round amplification. A PCR reaction system was as follows:

| | | |
|---|---|---|
| Ligated product | 2 | μL, |
| 10 × KOD buffer | 1.5 | μL, |
| dNTPs | 1 | μL, |
| MgSO₄ | 1 | μL, |
| U-F (10 μM) | 0.2 | μL, |
| gRNA-R (10 μM) | 0.2 | μL, |
| KOD-Neo-Plus | 0.3 | μL, |

| | |
|---|---|
| ddH₂O | supplemented to 15 μL; and |

Sequences of amplification primers U-F/gDNA-R in the first round of PCR were:

U-F (SEQ ID NO. 5):
5-CTCCGTTTTACCTGTGGAATCG-3;
and
gRNA-R (SEQ ID NO. 6):
5-CGGAGGAAAATTCCATCCAC-3.

Conditions of the first round PCR amplification were shown in Table 1:

TABLE 1

| | | | |
|---|---|---|---|
| 95° C. | 1 | min | |
| 95° C. | 10 | s | 10 cycles |
| 60° C. | 15 | s | |
| 68° C. | 20 | s | |
| 95° C. | 10 | s | 20 cycles |
| 60° C. | 15 | s | |
| 68° C. | 20 | s | |

After the amplification was completed, 3 μL of the amplified product was electrophoretically examined. A size of a target band was 564 bp.

The product obtained from the first round amplification was diluted 20-fold and used as a template for a second round amplification.

A reaction system of the second round PCR amplification was as follows:

| | | |
|---|---|---|
| First round amplified product | 1 | μL, |
| 10 × KOD buffer | 5 | μL, |
| dNTPs (2 mM) | 3 | μL, |
| MgSO₄ (25 mM) | 3 | μL, |
| Uctcg-B1′ (10 μM) | 0.2 | μL, |
| gRcggt-BL (10 μM) | 0.2 | μL, |
| KOD-Neo-Plus | 0.3 | μL, |

| | |
|---|---|
| ddH₂O | supplemented to 50 μL; and |

Sequences of second round PCR amplification primers were:

Uctcg-B1′ (SEQ ID NO. 7):
5′-TTCAGAggtctcTctcgCACTGGAATCGGCAGCAAAGG-3′;
and
gRcggt-BL (SEQ ID NO. 8):
5′-AGCGTGggtctcGaccgGGTCCATCCACTCCAAGCTC-3′.

Conditions of the second round PCR amplification were shown in Table 2:

TABLE 2

| | | | |
|---|---|---|---|
| 95° C. | 1 | min | |
| 98° C. | 10 | s | 25 cycles |
| 55° C. | 15 | s | |
| 68° C. | 20 | s | |
| 68° C. | 5 | min | |

After the amplification was completed, 3 μL of the amplified product was subjected to an electrophoresis, and a product length was examined. The second round amplified product was purified and its concentration was determined.
(5) Cleaving and ligating (two rounds of nested PCR amplification):

About 20 ng of the purified product obtained in step (4) was taken, about 20 ng of an uncleaved pYRCISPR/Cas9-MH plasmid was added, and a 15 μL reaction system was enzymatically digested with 10 U BsaI enzyme at 37° C. for 10 min.

After the enzymatic digestion, 1.5 μL of 10×T4 ligase buffer and 35U T4 ligase were added to the system. A PCR reaction system was shown in Table 3:

TABLE 3

| | | |
|---|---|---|
| 37° C. | 5 min | 25 cycles |
| 10° C. | 5 min | |
| 20° C. | 5 min | |

The ligated plasmid was transformed into E. coli by a heat shock method, and a positive clone was picked for detection. After sequencing was correct, the plasmid was sent to Wuhan Biorun biosciences Co., Ltd. for callus transformation. The rice OsPR6 gene knockout mutants OsPR6-1 and OsPR6-2 were obtained by taking Zhonghua 11 (ZH11) strain as a background.

### Example 2

Rice OsPR6 gene overexpression mutants were obtained.

According to a CDS sequence of OsPR6, primers OsPR6OX-F/R were designed. Primer sequences were as follows:

OsPR6OX-F (SEQ ID NO. 9):
gttacttctgcactaggtaccATGAACTCTACAAGTCATTTTGTCGC;
and
OsPR6OX-R (SEQ ID NO. 10):
tcttagaattcccggggatccTTAATTTAAAGCTATATATATTCTGCTA
GCTAGC.

By using a Zhonghua 11 (ZH11) genome as a template, the CDS sequence of the gene OsPR6 was amplified by using the primers OsPR6OX-F/R. After the amplified product was purified, the product was ligated to a pCAMBIA1390 vector (KpnI/BamHI double-digested) by a seamless cloning technique. The ligated plasmid was transformed into E. coli by a heat shock method, and a positive clone was picked for detection. After sequencing was correct, the plasmid was sent to Wuhan Biorun biosciences Co., Ltd. to be transformed to Agrobacterium and introduced into a rice callus for transformation. The rice OsPR6 gene overexpression mutants OXOsPR6-1 and OXOsPR6-2 were obtained by taking Zhonghua 11 (ZH11) strain as a background.

### Example 3

Effect of OsPR6 on disease resistance of rice.

In order to verify that a gene OsPR6 participates in a defense reaction of rice to rice blast, a resistance change of OsPR6 knockout mutants and overexpression mutants to rice blast was detected. A specific operation was as follows:

A wild Magnaporthe oryzae strain Guy11 was activated on an OA culture medium, and cultured in the dark at 25° C. for 3 days and in the light for 4 days. Sterile ddH₂O was added into a culture dish, a hypha was slightly scraped with an inoculating loop, Magnaporthe oryzae spores were eluted from a culture medium, and an eluent was filtered through a miracloth to obtain a spore suspension. The spore suspension was placed in a 2-mL centrifuge tube and centrifuged at 12,000 rpm for 2 min, a supernatant was discarded (bottom spores were prevented from being poured out), and sterilized ddH₂O was added to adjust a spore concentration to be more than or equal to 1×10⁶/mL. 1/10 volume of 0.1% Gelatin (a final concentration of Gelatin was 0.01%, v/v) was added to the spore solution for inoculation with Magnaporthe oryzae.

In-vitro inoculation: an in-vitro leaf was placed neatly on a surface of 2% water agar and spread flat, and a small amount of sterile ddH₂O was added to the culture dish to keep moisture. Surfaces of rice leaves (leaf veins were avoided) were slightly stirred by using a 10 μL sterilization pipette tip to damage the rice leaves, and perforation caused by an excessive force was avoided. 10 μL of the spore suspension was absorbed and dropped on a wound of the leaf and stood for 30 min. The culture dish was sealed by using a preservative film and placed in an incubator at 25° C. After culturing for 7 d, the inoculated leaves were photographed and the hypha biomass was counted.

Measurement of hypha biomass: disease spots of rice were cut and total DNA was extracted by a CTAB method. A rice gene Osubiquitin was used as an internal reference (LOC_Os03g13170), the amount of a Magnaporthe oryzae Mopot2 gene (MGG_13294) was detected, and the fungal biomass was analyzed through a real-time fluorescent quantitative PCR. A relative expression level of a gene was calculated by a 2^{-ΔΔCT} method. Primer sequences were:

qOsUBQ-F (SEQ ID NO. 11):
AAGAAGCTGAAGCATCCAGC;
qOsUBQ-R (SEQ ID NO. 12):
CCAGGACAAGATGATCTGCC;
Mopot2-F (SEQ ID NO. 13):
ACGACCCGTCTTTACTTATTTGG;
and
Mopot2-R (SEQ ID NO. 14):
AAGTAGCGTTGGTTTTGTTGGAT.

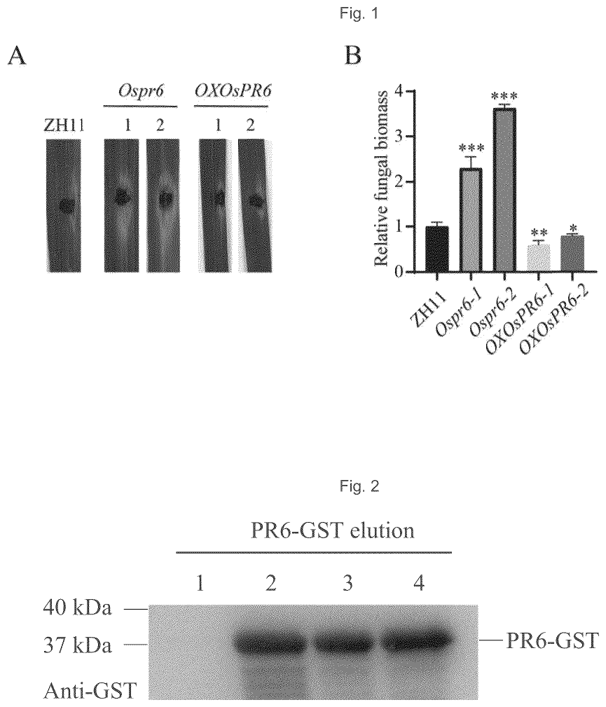

An in-vitro inoculation result (FIG. 1) showed that a disease spot area of the knockout mutants OsPR6-1 and OsPR6-2 was obviously larger than that of a wild type, and a rice blast resistance was obviously weakened; and the disease spot area of the overexpression mutants OXOsPR6-1 and OXOsPR6-2 was obviously smaller than that of the wild type, and the rice blast resistance was obviously enhanced, which indicates that OsPR6 was necessary for the rice blast resistance.

### Example 4

OsPR6 purified protein was obtained,

According a CDS sequence of a gene OsPR6, primers OsPR6GST-F/R were designed. Primer sequences were as follows:

OsPR6GST-F (SEQ ID NO. 15):
gatctggttccgcgtggatccAACTCTACAAGTCATTTTGTCGCCA;
and
OsPR6GST-R (SEQ ID NO. 16):
ctcgagtcgacccgggaattcTTAATTTAAAGCTATATATATTCTGCTA
GCTAGC.

Construction of vector: by using a Zhonghua 11 (ZH11) genome as a template, the CDS sequence of the gene OsPR6 was amplified by using the primers OsPR6GST-F/R. The amplified fragment was ligated to a PEGX-4T-1 vector by a seamless cloning technique. The ligated plasmid was transformed into E. coli by a heat shock method, and a positive clone was picked for detection.

Induction of PR6-GST protein expression: after the PR6-GST vector was sequenced correctly, the plasmid was transferred into an Escherichia coli strain BL21 by a heat shock method, a positive clone was picked, cultured at 37° C. until an OD value was 0.6, and induced at 16° C. for 16 h, an IPTG concentration was 1 mM, and then a protein expression was detected by Coomassie brilliant blue staining and Western-blot, so as to confirm that a protein induction was successful.

PR6-GST protein purification:

(1) every 150 mL of bacterial cells were centrifuged at 10,000 rpm for 2 min, and a supernatant was removed; 15 mL of a GST binding/washing buffer suspension was added to a precipitated bacterial block, and then 300 μL of 10 mg/mL lysozyme, 30 μL of 0.5 M MgCl₂, and 150 μL of 0.1 M phenylmethylsulfonyl fluoride (PMSF) were added. The mixture was slowly shaken at 4° C. for enzymolysis for 30 min.

(2) After the enzymolysis process was completed, the bacterial cells were ultrasonically crushed, and then are centrifuged at 4° C. for 10 min at a rotating speed of 10,000 rpm.

(3) 200 μL of a supernatant was taken and labeled as Input, the rest supernatant was transferred into an equilibrated GST resin flow column, a flow rate was adjusted to control at 8-10 s/drop, 200 μL of an effluent was collected and labeled as Flow through; 15 mL of water was added into a bacterial residue for suspension, and 200 μL of the suspension was labeled as Bacteria pellet; and a whole process was performed in a refrigerator at 4° C. to prevent protein denaturation.

(4) 5 mL of GST binding/washing buffer was added, the flow rate was controlled to be 3-5 s/drop, and 200 μL of an effluent below the column was collected and labeled as Wash1; and the step was repeated once, and 200 μL of an effluent was collected and labeled as Wash2.

(5) A proper amount of glutathione was added to 1 mL of GST elution buffer to elute a target protein, and an eluent (the eluent contains a target protein) was collected and labeled as Elution1; and the step was repeated 3 times, and the eluent was respectively labeled as Elution2, Elution3, and Elution4.

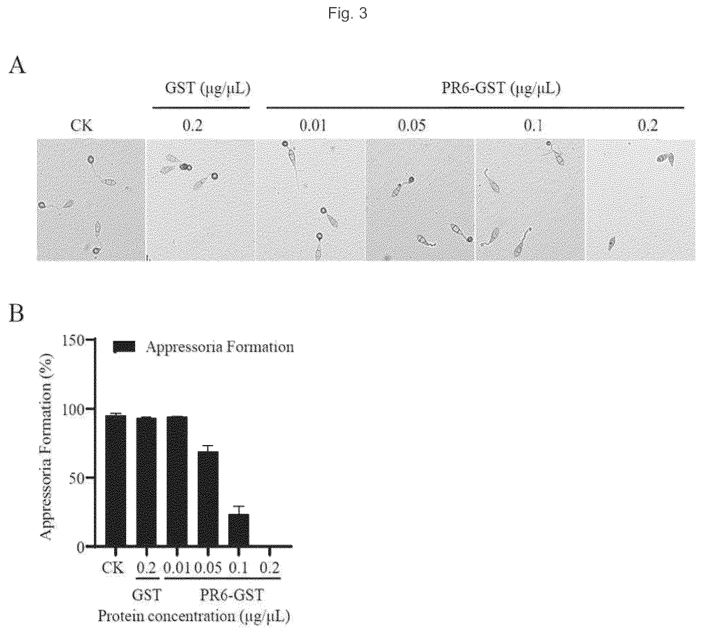

The purified protein was detected by Coomassie brilliant blue staining and Western-blot (FIG. 2), and a concentration of the purified protein was then determined.

### Example 5

An OsPR6 purified protein inhibited formation of appressorium of Magnaporthe oryzae.

A wild Magnaporthe oryzae strain Guy11 was activated on an OA culture medium, and the culture conditions and process of obtaining the spore suspension were the same as those in example 3. A spore concentration was adjusted to 1-2×10⁵/mL by using a hemocytometer, and concentrations of PR6-GST purified protein in the suspension were set at 0, 0.01, 0.05, 0.1, and 0.2 μg/μL respectively. The spore suspension was dropped on a surface of a hydrophobic slide to avoid agglomeration among water drops, and cultured at 25° C. for 16 h in a moisture environment. The formation of the appressorium of Magnaporthe oryzae was observed under a microscope.

As shown in FIG. 3, a PR6GST protein had an obvious inhibiting effect on the appressorium of Magnaporthe oryzae. When the concentration of the PR6-GST was 0.1 μg/μL, a formation rate of the appressorium of Magnaporthe oryzae was only 23.97%, and when the concentration of the PR6-GST reached 0.2 μg/μL, germination of Magnaporthe oryzae spores was extremely obviously inhibited, and no appressorium was formed. The result showed that the OsPR6 protein was expected to be used as a drug for preventing and treating rice blast.

What is claimed is:

1. A method for regulating resistance of rice to Magnaporthe oryzae, wherein when the resistance of rice to Magnaporthe oryzae needs to be reduced, silencing or knocking out an OsPR6 gene in rice; and when the resistance of rice to Magnaporthe oryzae needs to be improved, overexpressing the OsPR6 gene in rice.

2. A drug for resisting Magnaporthe oryzae, wherein an active component comprises a rice OsPR6 gene with a CDS nucleotide sequence shown in SEQ ID NO. 2 or a protein encoded by the rice OsPR6 gene with an amino acid sequence shown in SEQ ID NO. 1.

3. A method for constructing a transgenic rice resisting Magnaporthe oryzae, wherein a rice OsPR6 gene is transferred into a rice plant to obtain a transgenic rice with a high expression of the rice OsPR6 gene.

4. The method for constructing a transgenic rice resisting Magnaporthe oryzae according to claim 3, wherein comprises cloning a nucleotide sequence of a CDS region of the rice OsPR6 gene into a vector, and transferring the vector into Agrobacterium firstly, and then transferring the vector into a rice cell by a callus transformation to obtain a transgenic rice with a high expression of the rice OsPR6 gene.
